(19) United States
(12) Patent Application Publication
(10) Pub. No.: US 2008/0181957 A1
(43) Pub. Date: Jul. 31, 2008

(54) INCREASED AMORPHOUS STABILITY OF POORLY WATER SOLUBLE DRUGS BY NANOSIZING

(76) Inventors: Min Wei, Morris Plains, NJ (US); Shuqian Xu, Sunnyvale, CA (US); Andrew C. Lam, San Jose, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/767,393

(22) Filed: Jun. 22, 2007

Related U.S. Application Data
(60) Provisional application No. 60/816,293, filed on Jun. 23, 2006.

(51) Int. Cl. A61K 9/14; A61K 31/445

(52) U.S. Cl. 424/489; 514/317

(57) ABSTRACT

Disclosed is a population of nanoparticles, together with methods of making a population of nanoparticles, wherein one or more of the nanoparticles includes: an amorphous drug core having an effective diameter less than or equal to about 2.0 microns, wherein the amorphous drug core is substantially free of dopant, and wherein the amorphous drug core comprises a drug with properties that satisfy the following relationships: a glass transition temperature greater than or equal to about 30 Deg. C.; and water solubility at 25 Deg. C. less than or equal to about 1 mg/ml; and at least one stabilizer adsorbed on a surface of the amorphous drug core; and wherein the at least one stabilizer is present in an amount effective to provide an amorphous stability of the population of nanoparticles that is approximately equal to or greater than an amorphous stability of an amorphous bulk drug substance comprising the drug, as measured over a period of at least four months.

The present application is derived from and claims priority to provisional application U.S. Ser. No. 60/816,293, filed Jun. 23, 2006, which is herein incorporated by reference in its entirety.

### FIELD OF THE INVENTION

The present invention relates to methods and compositions that provide improved solubility of poorly soluble drugs. More particularly, the invention relates to populations of nanoparticles and related methods that provide improved solubility of poorly soluble drugs.

### DESCRIPTION OF THE RELATED ART

Lead compounds that are currently being developed using combinatorial chemistry and other high throughput techniques often demonstrate very poor solubility. This may be in part because pharmaceutical companies may choose to screen first for activity against a target, and only then for pharmacokinetic properties. This can lead to discovery of very active compounds that are not particularly good orally dosed drugs.

If new drug leads have poor solubility, this may lead to poor oral absorption from the gastrointestinal tract. Poor oral absorption leads to poor bioavailability, and consequently poor drug performance.

These problems have been recognized in the industry. See M. Kataoka et al., “In Vitro System to Evaluate Oral Absorption of Poorly Water-Soluble Drugs Simultaneous Analysis on Dissolution and Permeation of Drugs,” Pharm. Res. 20(10):1674-1680 (2003).

Development of new technologies to improve solubility has generated scientific interest, resulting in a large array of new systems that can be applied to compounds with intrinsically low solubility with associated poor dissolution performance. K. R. Horspool et al., “Advancing new drug delivery concepts to gain the lead.” Drug Delivery Technology 3:34-46 (2003) (“Horspool”). Horspool goes on to say:
“Many of the systems have been designed to overcome solubility issues associated with high lipophilicity. However, problems remain with solubility associated with highly crystalline materials that exhibit strong intermolecular interactions and a high propensity to crystallize. This issue is exacerbated because discovery screening typically involves testing of amorphous forms of compounds in dimethyl sulphoxide (DMSO). Testing of these low-energy forms facilitates candidate selection based primarily on efficacy considerations with minimal regard to future complications due to changes to the bulk form. Solubility problems can arise later in development when the drug substance synthetic process is scaled and a highly crystalline, insoluble form is isolated. Compounds with high crystal lattice energy can pose significant solubility problems that cannot be addressed with technologies designed to overcome lipophilicity issues. We estimate that between 10% and 30% of hits identified in high throughput screens could have latent solubility issues associated with crystal packing that would not be predicted based on lipophilicity. Technologies, such as size reduction to nanoparticles (Elan, Skyepharma, Baxter) and stabilization of amorphous forms (SOLIQS), offer options, but these approaches may not always be the answer because of the tendency of some materials to undergo physical changes. Development of alternate systems to address this specific issue is worthy of further investment by DD providers and pharma companies with due consideration of the supply versus demand to avoid development of “excess capacity” and poor adoption of a large number of new technologies.”

U.S. Pat. No. 5,145,684 to Liversidge et al. discloses crystalline nanoparticles having a surface modifier adsorbed onto the surface of the nanoparticles. This patent does not disclose amorphous nanoparticles.

U.S. Pat. No. 6,656,504 to Bosch et al. and U.S. Published Patent Application 2002/0016290 to Floc'h et al. disclose nanoparticulate amorphous cyclosporine formulations. However, cyclosporine takes on an amorphous form quite easily and doesn't have a very stable crystalline form. This property is in contrast to most other poorly water-soluble drugs. The glass forming ability (GFA) of cyclosporine is greater than about 0.85.

Amorphous nanoparticles are disclosed in K. Chari et al., Effect of Poly(vinylpyrrolidone) on Transformation of the Dispersed Phase and Gelation in a Lyophobic Colloid System, J. Phys. Chem. 97:2640-2645 (1993) (“Chari 1”), and K. Chari et al., Dispersed Phase Microstructure in a Colloid Gel, J. Phys. Chem. 98:5125-5126 (1994) (“Chari 2”). However, these nanoparticle populations are not very stable, with changes apparent after 20 days in Chari 1, and after one week in Chari 2.

Amorphous nanoparticles are also disclosed in K. Chari et al., Polymer-Surfactant Interaction and Stability of Amorphous Colloidal Particles, J. Phys. Chem. B. 103:9867-9872 (1999). While the paper shows data that suggest that the size of the nanoparticles may remain relatively stable over one year, there is no evidence presented that the nanoparticles actually retain their amorphous stability over the year.

Although amorphous nanoparticles can be obtained by precipitation, the stability of amorphous nanoparticles made by this method is still fundamentally unsolved because of the impurities (dopants) and defects in the particles. B. Rabinow, Nanosuspensions in Drug Delivery, Nature Rev. Drug Discovery 3:785-796 (2004).

Accordingly, substances, compositions, dosage forms and methods that address the above noted problems in the art are needed.

### BRIEF SUMMARY OF THE INVENTION

In an aspect, the invention relates to a population of nanoparticles wherein one or more of the nanoparticles comprises: an amorphous drug core having an effective diameter less than or equal to about 2.0 microns, wherein the amorphous drug core is substantially free of dopant, and wherein the amorphous drug core comprises a drug with properties that satisfy the following relationships: a glass transition temperature greater than or equal to about 30 Deg. C.; and water solubility at 25 Deg. C. less than or equal to about 1 mg/ml; and at least one stabilizer adsorbed on a surface of the amorphous drug core; and wherein the at least one stabilizer is present in an amount effective to provide an amorphous stability of the population of nanoparticles that is approximately equal to or greater than an amorphous stability of an amorphous bulk drug substance comprising the drug, as measured over a period of at least four months.

In another aspect, the invention relates to a method of making a population of nanoparticles comprising: forming amorphous drug cores with an effective diameter less than or equal to about 2.0 microns, wherein the amorphous drug cores are substantially free of dopant, and wherein the amorphous drug cores comprise a drug with properties that satisfy the following relationships: a glass transition temperature greater than or equal to about 30 Deg. C.; and water solubility at 25 Deg. C. less than or equal to about 1 mg/ml; and adsorbing at least one stabilizer on a surface of the amorphous drug cores; wherein the at least one stabilizer is present in an amount effective to provide an amorphous stability of the population of nanoparticles that is approximately equal to or greater than an amorphous stability of an amorphous bulk drug substance comprising the drug, as measured over a period of at least four months.

### BRIEF DESCRIPTION OF THE SEVERAL VIEWS OF THE DRAWINGS

FIG. 1 shows XRD spectra of bulk amorphous COMPOUND 1 after 3 month (lower) and 1 year (upper) storage.

FIG. 2. shows XRD spectra of nanosized amorphous COMPOUND 1 after 3 month (lower) and 1 year (upper) storage.

FIG. 3 shows bulk amorphous COMPOUND 1 stability at 0 month, 1 month, 2 month, 4 month (crystallinity: 0.44%), 5 month (crystallinity: 1.39%), and 6 month (crystallinity: 10.54%) time points (from lower to upper) examined by DSC.

FIG. 4 shows nanosized amorphous COMPOUND 1 stability at 0 month, 2 month, 4 month, 6 month, 8 month, 10 month and 12 month time points (from lower to upper) examined by DSC.

FIG. 5 shows XRD of nanosized amorphous COMPOUND 1 after 0 week (lower) and 8 week (upper) storage.

FIG. 6 shows particle size of nanosized amorphous COMPOUND 1 in 8 weeks storage at 25° C.

FIG. 7 shows XRD of the as milled nanosized amorphous drug in aqueous suspension (7.5% drug loading) (top) and in diluted aqueous nanosuspension with 2.0% (middle) and 1.0% (bottom) drug loading (diluted with deionized water.

FIG. 8 shows XRD of nanosized amorphous terfenadine after 3 month (lower) and 1 year (upper) storage.

FIG. 9 shows bulk amorphous terfenadine stability at 0 month, 2 month, 4 month, 6 month, and 8 month time points (from lower to upper) examined by DSC.

FIG. 10 shows nanosized amorphous terfenadine stability at 0 month, 2 month, 4 month, 6 month, and 8 month time points (from lower to upper) examined by DSC.

### DETAILED DESCRIPTION OF THE INVENTION

### 1. Introduction

The inventors have surprisingly found that the problems noted above can be solved by providing a population of nanoparticles, and methods of making such populations of nanoparticles, wherein one or more of the nanoparticles comprises: an amorphous drug core having an effective diameter less than or equal to about 2.0 microns, wherein the amorphous drug core is substantially free of dopant, and wherein the amorphous drug core comprises a drug with properties that satisfy the following relationships: a glass transition temperature greater than or equal to about 30 Deg. C.; and water solubility at 25 Deg. C. less than or equal to about 1 mg/ml; and at least one stabilizer adsorbed on a surface of the amorphous drug core; and wherein the at least one stabilizer is present in an amount effective to provide an amorphous stability of the population of nanoparticles that is approximately equal to or greater than an amorphous stability of an amorphous bulk drug substance comprising the drug, as measured over a period of at least four months.

The value of the present invention can be seen by reference to the Examples. For instance, in Example 1, differential scanning calorimetry studies showed recrystallization of amorphous bulk drug substance after 4 month of storage at 25 Deg C., whereas no recrystallization was detected for populations of inventive nanoparticles during a time period of 1 year. This is very significant, because it represents an unexpected result: given the higher energy state of amorphous nanoparticles, as compared to amorphous bulk drug substance, one of skill would have expected the inventive populations of nanoparticles to exhibit less amorphous stability, not more. Example 2 suggests that aqueous suspensions of populations of inventive nanoparticles can retain their amorphous stability, and mean particle size, for 8 weeks storage at 25° C., which is a very significant result because water can greatly accelerate the recrystallization of amorphous drugs (B. C, Hancock et al, The Relationship between the Glass Transition Temperature and the Water Content of Amorphous Pharmaceutical Solids. Pharm. Res. 11:471-477 (1997). This result further supports the notion that the inventive population of nanoparticles, and related methods, are relatively stable in their amorphous character. Example 3 further supports the unexpected nature of the present invention, as it shows another drug that exhibits improved amorphous stability as a population of inventive nanoparticles (along with related methods) as compared to the amorphous bulk drug substance from which it was made.

All of these advantages represent significant improvements over the art.

The invention, and embodiments thereof, will now be described in more detail.

### 2. Definitions

All percentages are weight percent unless otherwise noted.

All references cited herein are incorporated by reference in their entirety and for all purposes to the same extent as if each individual publication or patent or patent application was specifically and individually indicated to be incorporated by reference in its entirety for all purposes. The discussion of references herein is intended merely to summarize the assertions made by their authors and no admission is made that any reference constitutes prior art. Applicants reserve the right to challenge the accuracy and pertinence of the cited references.

The present invention is best understood by reference to the following definitions, the drawings and exemplary disclosure provided herein.

“Adsorbed” or “adsorption” means accumulated or accumulation on a surface of a solid, such as an amorphous drug core.

“Amorphous bulk drug substance” means a portion of a drug being larger than sub-micron in size and having generally amorphous properties. Methods of forming bulk drug substance are found elsewhere herein.

“Amorphous drug core” means a central portion of an inventive nanoparticle that comprises one or more drugs in a substantially amorphous state. An inventive amorphous drug core is substantially amorphous on its surface and in its interior. Accordingly, inventive populations of nanoparticles can be distinguished from populations of crystalline nanoparticles that may have an amorphous surface, due perhaps to the method of preparing such crystalline nanoparticles, but contain a highly crystalline interior. Of course, crystalline nanoparticles that have a crystalline surface and interior are also distinguishable from the inventive nanoparticles.

The amorphous state of the amorphous drug core is determined by subjecting a population of nanoparticles that comprise one or more nanoparticles that comprise the recited amorphous drug core to differential scanning calorimetry (DSC). In an embodiment, a DSC method according to the invention used a Perkin-Elmer DSC-7 or a Diamond DSC calorimeter applied for the measurement of specific transition temperatures of tested samples. Thus, the glass transition (Tg), melting (Tm) and crystallization (Tc) temperatures were measured for each sample, according to the PN-EN ISO 11357-1:2002 (ISO 11357-1:1997), ISO 11357-2:1999 and ISO 11357-3:1999 standards, at the rate of temperature change of 10 Deg C./min. The instrument was calibrated using indium, tin and zinc certified reference materials (CRMs)

The amorphous state of the amorphous drug core, as measured by DSC is expressed as a weight fraction of the weight of amorphous material in the amorphous drug core to the total weight of the amorphous drug core, expressed as an average value across the population of nanoparticles being measured. For instance, if a population of nanoparticles was measured using DSC, and the amorphous state was determined to be a particular value for the population, that value would be considered the average amorphous state for each nanoparticle within the population. An inventive amorphous drug core may contain a small amount of crystalline drug. In an embodiment, the amorphous drug core is substantially amorphous, preferably at least about 95% w/w amorphous, still more preferably at least about 98% w/w amorphous, even more preferably at least about 99% w/w amorphous, yet more preferably at least about 99.5% w/w amorphous, and most preferably at least about 99.9% w/w amorphous.

“Amorphous stability” means a measure of how much a material changes in its structure from being amorphous to being crystalline under defined conditions and a set timeframe. Amorphous materials such as a population of nanoparticles has amorphous stability if the weight fraction of amorphous material to total weight of the amorphous drug core changes by less than 10 percent, on an absolute basis, over 6 months at 25 degree C. For instance, an amorphous material that is initially 98% w/w amorphous is considered amorphously stable according to the invention if, at the end of 6 months testing at 25 degree C., the material is at least 88% w/w amorphous. The amorphous state of a material according to the invention is determined using a DSC method as detailed above and elsewhere herein.

In an embodiment, inventive nanoparticles exhibit greater than about 6 months stability, more preferably greater than about 9 months stability, still more preferably greater than about 12 months stability, yet more preferably greater than about 18 months stability, even more preferably greater than about 24 months stability.

“Dopant” means one or more substances added to another material in order to affect a physical property of the other material. The present invention discloses amorphous drug cores that are substantially free of dopant. In a preferred embodiment, the amorphous drug cores that are substantially free of dopant comprise amorphous drug cores containing less than about 15 weight percent dopant, more preferably less than about 10 weight percent dopant, still more preferably less than about 5 weight percent dopant, and yet more preferably less than about 1 weight percent dopant; all weight percentages being based on the total weight of the amorphous drug core.

“Drug(s)” means one or more biologically active substances that are useful or potentially useful in the treatment of various diseases, disorders, and the like. In a preferred embodiment, drugs useful in the practice of the invention comprise those drugs that fall in Biopharmaceutics Classification System (BCS) classes II and IV.

“Effective diameter” means a value such that at least 50% of a particle population has a weighted average particle size of less than the value, with the particle size measured using particle size measurement techniques known in the art. Effective diameter may be determined using a particle sizer, including but not limited to dynamic light scattering, laser light diffraction/scattering, atomic force microscopy (AFM), transmission electron microscopy (TEM), or scanning electron microscopy (SEM). In an embodiment, the effective diameter of an amorphous drug core according to the invention is less than or equal to about 2.0 microns, preferably less than or equal to about 1.5 micron, more preferably less than or equal to about 1.0 micron, and still preferably less than or equal to about 0.75 micron.

“Glass transition temperature” or “Tg” means that temperature at which a material transitions to a glassy state from a liquid state, as measured at standard atmospheric pressure. Drugs useful in the practice of the invention comprise those drugs having a glass transition temperature greater than or equal to about 50 Deg. C. Preferably, the drugs have a Tg greater than or equal to about 60 Deg. C., more preferably the drugs have a Tg greater than or equal to about 70 Deg. C., still more preferably the drugs have a Tg greater than or equal to about 80 Deg. C., and yet more preferably the drugs have a Tg greater than or equal to about 100 Deg. C.

“Melting temperature” or “Tm” means the temperature at which the solid drug becomes a liquid at 1 atmosphere pressure.

“Stabilizer” means one or more substance(s) that are effectively adsorbed to a surface of an amorphous drug core but do not chemically bond to the amorphous drug core. In an embodiment, the adsorption of stabilizer on the amorphous drug core is in an amount sufficient to maintain an effective diameter of an amorphous drug core less than or equal to about 2.0 microns, preferably less than or equal to about 1.5 micron, more preferably less than or equal to about 1.0 micron, and still preferably less than or equal to about 0.75 micron. Preferably, the stabilizer may be an amorphous material (either in solid or in solution) by itself, and may in certain embodiments have some hydrophobic group(s) in the chemical structure. Suitable surface stabilizers are preferably selected from known organic and inorganic pharmaceutical excipients (GRAS). Such excipients include various polymers, low molecular weight oligomers, natural products, and surfactants. Preferred surface stabilizers are hydrophilic nonionic polymer or copolymers with one or more weak polar group(s). Combinations of different stabilizers and or co-stabilizers may be useful in the practice of this invention.

In a preferable embodiment, stabilizers may comprise co-stabilizers. Co-stabilizers comprise nonionic or ionic surfactants or polymers, which cannot effectively stabilize the particles in the absence of stabilizers. However, in presence of a stabilizer, a co-stabilizer can significantly improve stabilization of stabilizers by enhancing static repulsion and/or playing a role of Ostwald ripening inhibitor and/or recrystallization inhibitor. Preferred co-stabilizers are those that are not prone to solubilize the drug, such as double chain ionic surfactants.

The surface stabilizers and co-stabilizers employed in the present invention can be polymers or copolymers; surfactants, peptides and/or proteins and combinations thereof. Representative examples of surface stabilizers and co-stabilizers include polymer or copolymers, surfactants, proteins and other pharmaceutical excipients listed in Handbook of Pharmaceutical Excipients, published jointly by the American Pharmaceutical Association and The Pharmaceutical Society of Great Britain (The Pharmaceutical Press, 1986), such as:

Polyvinylpyrrolidone (e.g. PVP K12, PVP K17, and PVP K30 etc.)

Cellulosic polymers, such as HPC-SL, HPC-L, HPMC

Copolymer of Vinyl Pyrrolidone and Vinyl acetate (e.g. Plasdone® S630, VA64)

Poloxamers, such as, Pluronics® F68, F108 which are block copolymers of ethylene oxide and propylene oxide);

Polyethylene Glycol (e.g. PEG 400, PEG 2000, PEG 4000, etc)

Polyvinyl alcohol (PVA),

Tyloxapol

Polyoxyethylene Castor oil Derivatives

Colloidal silicon Dioxide

Carbomers (e.g. Carbopol 934 (Union Carbide); CMC Na

Polysobate 80, 20 etc.

Benzalkonium chloride

Charged Phospholipids

Sodium Docusate, Aerosol OT (Cytec)

Others examples include gelatin, casein, lysozyme, albumin, cholesterol, stearic acid, calcium stearate, glycerol monostearate, sodium dodecylsulfate, methylcellulose, noncrystalline cellulose, magnesium aluminium silicate, Triton® X-200 (an alkyl aryl polyether sulfonate available from Rohm and Haas). Mixtures of any of the above are also within the scope of the invention.

“Nanoparticle” means a particle having an effective diameter less than or equal to about 2.0 microns, preferably less than or equal to about 1.5 micron, more preferably less than or equal to about 1.0 micron, and still preferably less than or equal to about 0.75 micron.

“Nanosizing the amorphous bulk drug substance” means forming amorphous drug cores that, in an embodiment, possess effective diameters less than or equal to about 2.0 microns, preferably less than or equal to about 1.5 micron, more preferably less than or equal to about 1.0 micron, and still preferably less than or equal to about 0.75 micron. Methods of nanosizing the amorphous bulk drug substance are found elsewhere herein.

“Water solubility” means a measure of the maximum possible concentration of a drug dissolved in water. The water temperature may be specified; in an embodiment water solubility is determined at 25 Deg. C. Units of measurement of water solubility are typically mass/volume, such as mg/ml. The water solubility of drug useful in the practice of the present invention is less than or equal to about 1 mg/ml at 25 Deg. C.; preferably less than or equal to about 0.1 mg/ml at 25 Deg. C.; more preferably less than or equal to about 0.01 mg/ml at 25 Deg. C., still more preferably less than or equal to about 1 microgram/ml at 25 Deg. C.

### 3. Materials and Methods for Making the Inventive Nanoparticles

The inventive nanoparticles may be made by a variety of methods, as generally set forth herein.

Amorphous bulk drug substances according to the invention may be formed in a variety of ways including but not limited to directly obtaining through chemical synthesizing, melting/quenching the drug, solvent casting the drug, super critical fluid extraction, rapid precipitation by antisolvent addition, grinding/milling, freeze drying, spray freezing (e.g. Enhanced aqueous dissolution of a poorly water soluble drug by novel particle engineering technology: spray-freezing into liquid with atmospheric freeze-drying. Pharm Res. 2003 March; 20(3):485-93), solvent extraction, or dehydration of hydrated compounds (e.g. Advanced Drug Delivery Reviews 48 (2001)27-42), freeze-drying, spray-drying (e.g. J. Broadhead, S. K. Rouan Edmond, C. T. Rhodes, The spray drying of pharmaceuticals, Drug Dev. Ind. Pharm. 18 (1992) 1169-1206.), or combinations of the above. Additional methods may be found in Yu L., Amorphous pharmaceutical solids: preparation characterization and stabilization. Adv. Drug. Delivery Rev., 2001, 48, p. 27-42.

Typically, the method or methods of forming amorphous bulk drug substances according to the invention will result in amorphous bulk drug substances that are substantially amorphous, preferably at least about 80% w/w amorphous, more preferably at least about 85% w/w amorphous, still more preferably at least about 90% w/w amorphous, even more preferably at least about 95% w/w amorphous, yet more preferably at least about 99% w/w amorphous, and most preferably at least about 99.5% w/w amorphous. The weight fraction of amorphous material in the amorphous bulk drug substance may be determined according to the DSC methods disclosed herein as being useful for determining weight fraction of amorphous material in the inventive amorphous drug cores. When preparing bulk amorphous drug, it is preferred that there is no excipient and/or dopant added.

Amorphous bulk drug substances according to the invention may be nanosized in a variety of ways, including but not limited to milling (as described, for example, in U.S. Pat. No. 5,145,684), high speed homogenization, hydrodynamic cavitation (as described, for example, in U.S. Pat. No. 5,858,410), ultrasonication (as described, for example, in U.S. Pat. No. 5,091,188), or combinations of any of the above methods. Operation at relatively low temperatures and pressures is preferred. For example, the size reduction operation temperature is preferred to be done at temperatures at least 10 Degree C. lower than the drug's Tg. Atmospheric pressure is preferred during nanosizing operations.

A typical effective diameter target for a nanosizing operation according to the invention is to get the effective diameter of particles to be equal to or less than about 0.8 micron. The particle size can be checked during or after the nanosizing operation. If particle size doesn't decrease even if the nanosizing time is extended, the operation is essentially complete, or must be continued using a different unit operation.

Preferably, stabilizers, and optional co-stabilizers, may be combined with the amorphous bulk drug substances prior to nanosizing the amorphous bulk drug substances. In certain embodiments, stabilizers and optional co-stabilizers may be added during or shortly after the nanosizing. The timing of adding the stabilizers may be dependent on interactions between the amorphous bulk drug substance and the particular stabilizer and optional co-stabilizer. In embodiments, the weight ratio of amorphous bulk drug substance to stabilizer (including optional co-stabilizer) ranges from about 1/2 to about 20/1, preferably from about 1/1 to about 10/1. Preferably, the weight ratios are measured based on the amorphous bulk drug substance to stabilizer (including optional co-stabilizer) added to the nanosizing operation (as opposed to direct measurement of the inventive nanoparticles themselves.

While there has been described and pointed out features and advantages of the invention, as applied to present embodiments, those skilled in the medical art will appreciate that various modifications, changes, additions, and omissions in the method described in the specification can be made without departing from the spirit of the invention. In particular, the following Examples are intended to be illustrative, and not limiting in any way, of the present invention.

### 4. Examples

### Example 1

The following drug (COMPOUND 1), with a water solubility less than or equal to about 0.2 ng/ml, was selected for forming nanoparticles according to the invention.

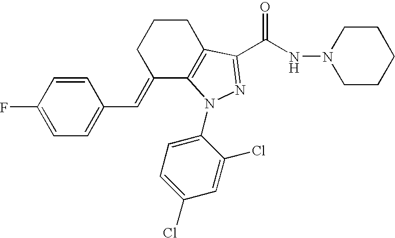

After the crystalline form of this drug was melted at 200° C. in an aluminum container, it was quickly transferred into an ice bath and converted into amorphous bulk drug substance. The amorphous bulk drug substance was then mixed with water, stabilizers and other milling media. The mixture was loaded into a mechanical mill (Elan, Nanomill). Shear force was applied by milling to nanosize the bulk amorphous drug into nanosized amorphous drug-comprising particles with adsorbed stabilizer, i.e. the inventive population of nanoparticles. The nanosized formulations were collected, and dried through lyophilization if necessary.

Composition for wet milling, expressed as weight percent based on total weight of material charged to the mill.

| | |
|---|---|
| Compound 1: | 15% |
| Hydroxypropylmethyl cellulose (HPMC): | 3.5% |
| Dioctyl Sodium Sulfosuccinate (USP, Cytec, Inc): | 0.25% |
| Deionized water: | 81.25% |
| Total: | 4.64 g |

Conditions for Milling:

| | |
|---|---|
| Milling media: | 5.43 g Polymill 500 (Elan) |
| Temperature: | 6.0 ± 0.2 Deg C. |
| Speed: | 5500 ± 200 rpm |
| Milling Volume: | 10 cc |

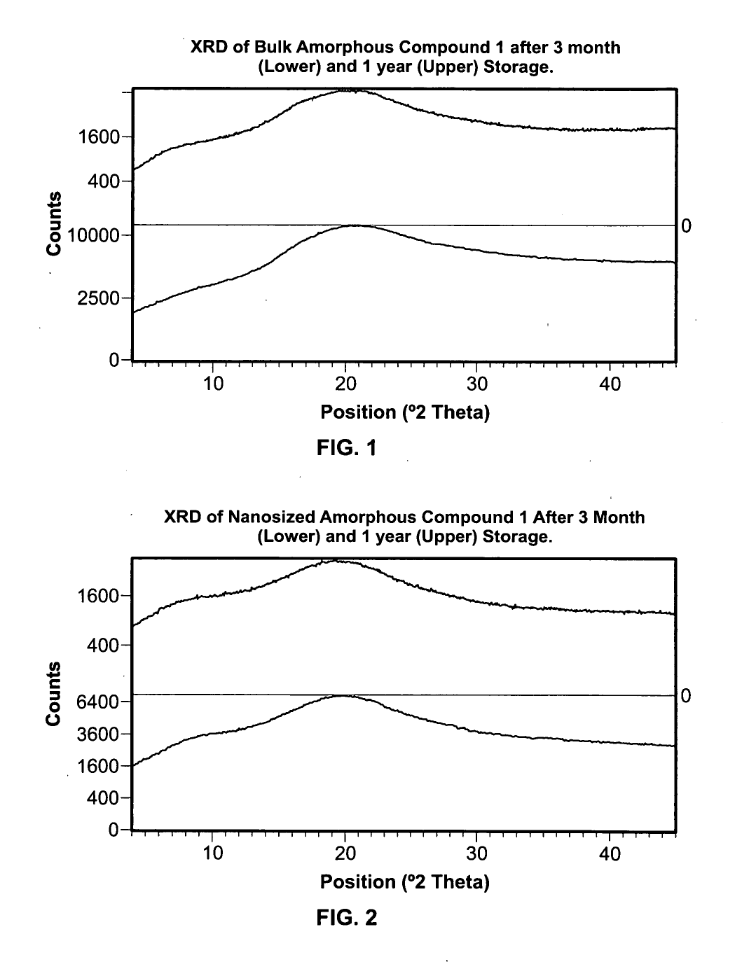

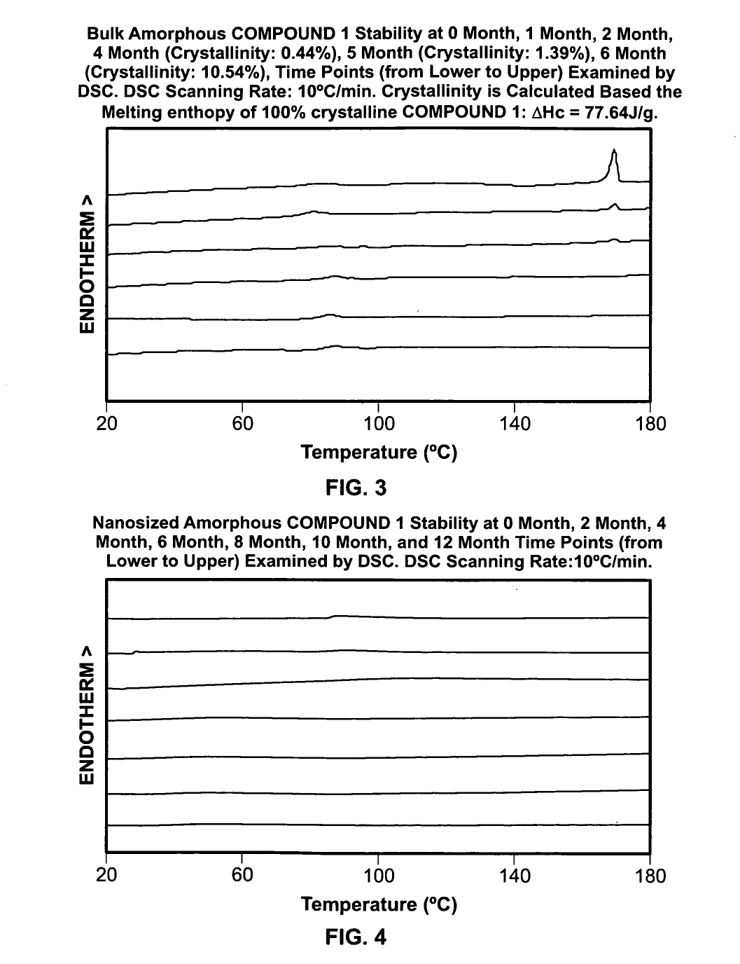

After milling, the mean particle size of nanosized amorphous drug was 474 nm as measured on a Horiba—910 light scattering particle sizer. The X-ray diffraction spectra (XRD) (FIGS. 1 and 2) show that in both bulk and nanosized amorphous COMPOUND 1 (after drying by lyophilization) samples, XRD amorphous stability can be achieved up to 1 year. However, DSC studies in FIG. 3 show that the recrystallization of amorphous COMPOUND 1 in bulk was detectable after 4 month of storage at 25 Deg C., though the crystallinity was relatively low. In contrast, no recrystallization was detected in the DSC studies for nanosized amorphous COMPOUND 1 within time period of 1 year (FIG. 4). This comparison demonstrates that the amorphous stability of COMPOUND 1 can be enhanced by practicing the present invention.

### Example 2

The preparation of Example 1 was substantially duplicated, except for the following changes:

Composition for wet milling, expressed as weight percent based on total weight of material charged to the mill.

| | |
|---|---|
| Compound 1: | 7.5% |
| Hydroxypropylmethyl cellulose (HPMC): | 3.8% |
| Dioctyl Sodium Sulfosuccinate (USP, Cytec, Inc): | 0.27% |
| Deionized water: | 88.43% |
| Total: | 4.64 g |

Condition of Milling:

| | |
|---|---|
| Milling media: | 5.43 g Polymill 500 (Elan) |
| Temperature: | 6.0 ± 0.2 Deg C. |
| Speed: | 5500 ± 200 rpm |
| Milling Volume: | 10 cc |

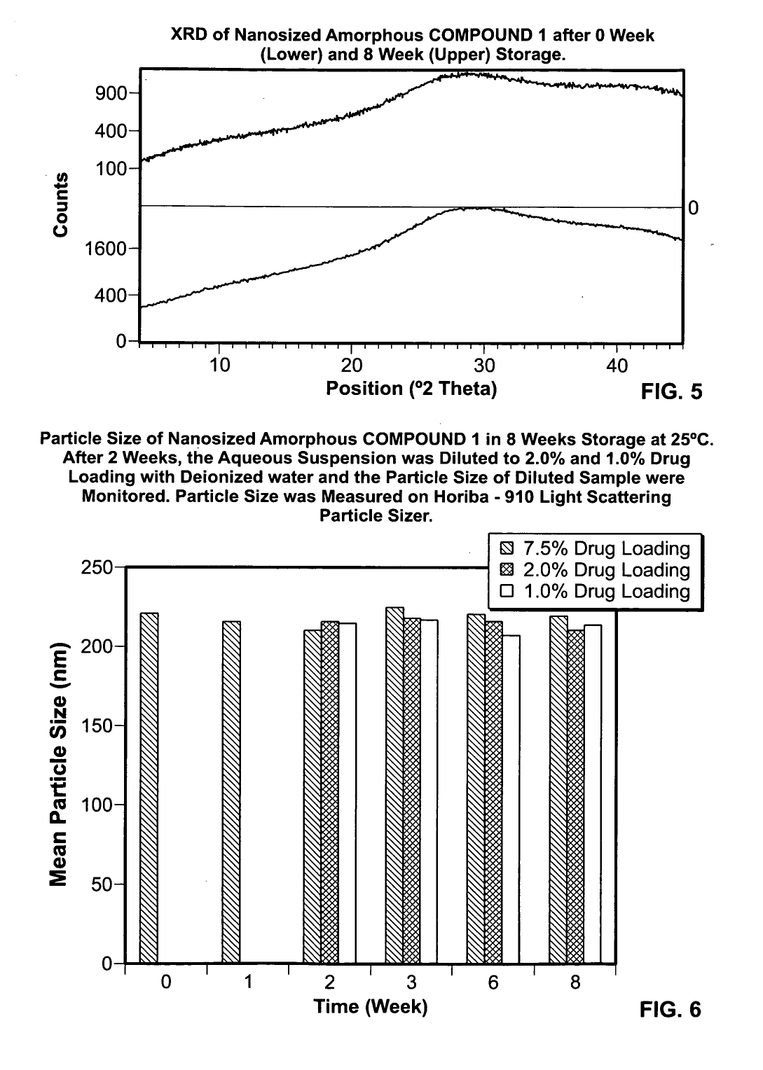

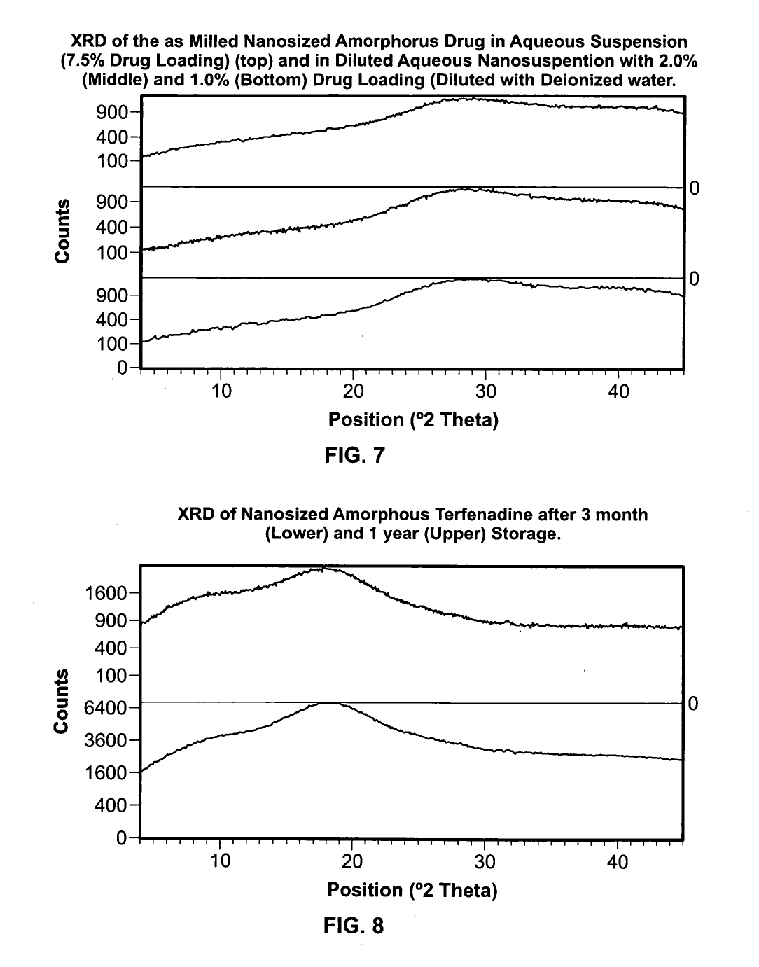

After size reduction by milling, inventive amorphous drug nanoparticles were obtained. In this example, the mean particle size of inventive nanoparticles comprising amorphous drug cores that comprise COMPOUND 1 was about 200 nm. Scanning Electron Microscopy (SEM) microphotographs of the inventive nanosized amorphous drug, suggest a size range of less than 500 nm for individual nanoparticles. Moreover, the nanoparticless don't have regular shape that most crystalline particles have, indicating the particles are in amorphous state. The phase behavior of nanosized amorphous COMPOUND 1 in aqueous suspension before and after 8 weeks storage at 25° C. was monitored by XRD (FIG. 5). Because water can function as a plasticizer to decrease the amorphous stability of poorly water soluble drugs, obtaining amorphous stability for the inventive nanosized amorphous drug may be quite challenging. XRD results for the nanosized amorphous COMPOUND 1 in aqueous suspension show that there is no diffraction peaks for nanosized amorphous COMPOUND 1, even after 8 weeks storage in aqueous environment, which is a significant result. FIG. 6 shows the particle size stability of the inventive nanosized amorphous drug. It is shown that not only phase behavior but also particle size doesn't change over the 8 week test period, even with further dilution. FIG. 7 shows that the amorphous property is also maintained upon dilution.

### Example 3

### Terfenadine

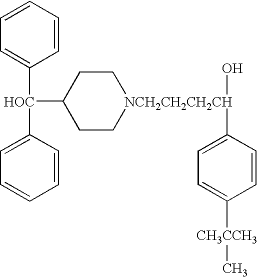

Terfenadine is an antihistamine drug. After the crystalline form of this drug was melted at 170° C. and quickly transferred into dry ice (solid CO₂) bath and converted into amorphous bulk drug substance, and was mixed with water, stabilizers and other milling media. The mixture was loaded into a mechanical mill. Shear force was applied to nanosize the amorphous bulk drug substance into nanosized amorphous drug with adsorbed stabilizer, i.e. the inventive population of nanoparticles. The formulations were collected, and dried through lyophilization if necessary.

Composition for wet milling, expressed as weight percent based on total weight of material charged to the mill.

| | |
|---|---|
| Terfenadine: | 5% |
| Hydroxypropylmethyl cellulose (HPMC): | 2.85% |
| Dioctyl Sodium Sulfosuccinate (USP, Cytec, Inc): | 0.14% |
| DI water: | 92.01% |
| Total: | 4.64 g |

Condition of Milling:

| | |
|---|---|
| Milling media: | 5.44 g Polymill 500 (Elan) |
| Temperature: | 6.0 ± 0.2° C. |
| Speed: | 5500 ± 200 rpm |
| Milling Volume: | 10 cc |

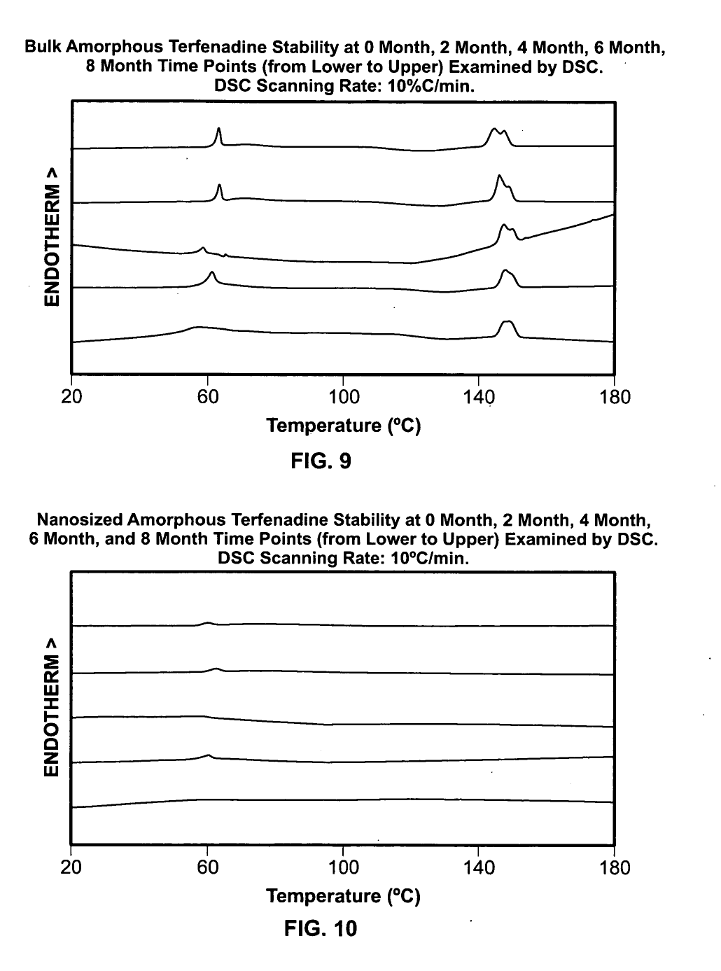

From the DSC study, it was found that crystalline terfenadine has a melting peak at 151.06° C. and no glass transition was detected. After melting and quenching, the amorphous terfenadine has a glass transition at 53.77° C. (Tg) but also has a small melting peak at 148.89° C. After nanosizing by milling, inventive nanoparticles were obtained having a mean particle size of 374 nm, which was measured using Horiba—910 light scattering particle sizer. An XRD (FIG. 8) study shows that the crystallinity of nanosized amorphous terfenadine has been reduced to an undetectable level. FIG. 9 shows the amorphous stability of bulk amorphous terfenadine. It can be illustrated that there is recrystallization happening in the bulk amorphous terfenadine, indicated by a melting peak at ˜148° C. FIG. 10 shows stability data of nanosized amorphous terfenadine; it can be seen that the nanosized amorphous terfenadine did not show recrystallization after 8 month storage at 25 Deg C. Again, these results about terfenadine demonstrate that the amorphous stability is enhanced after practicing the present invention.

What is claimed is:

1. A population of nanoparticles wherein one or more of the nanoparticles comprises:
an amorphous drug core having an effective diameter less than or equal to about 2.0 microns, wherein the amorphous drug core is substantially free of dopant, and wherein the amorphous drug core comprises a drug with properties that satisfy the following relationships:
a glass transition temperature greater than or equal to about 30 Deg. C.; and
water solubility at 25 Deg. C. less than or equal to about 1 mg/ml; and
at least one stabilizer adsorbed on a surface of the amorphous drug core; and
wherein the at least one stabilizer is present in an amount effective to provide an amorphous stability of the population of nanoparticles that is approximately equal to or greater than an amorphous stability of an amorphous bulk drug substance comprising the drug, as measured over a period of at least four months.

2. The population of nanoparticles of claim 1 wherein the amorphous drug core comprises an amorphous drug core that is substantially amorphous.

3. The population of nanoparticles of claim 2 wherein the amorphous drug core comprises an amorphous drug core that is at least about 95% w/w morphous.

4. The population of nanoparticles of claim 3 wherein the amorphous drug core comprises an amorphous drug core that is at least about 98% w/w amorphous.

5. The population of nanoparticles of claim 4 wherein the amorphous drug core comprises an amorphous drug core that is at least about 99% w/w amorphous.

6. The population of nanoparticles of claim 5 wherein the amorphous drug core comprises an amorphous drug core that is at least about 99.5% w/w amorphous.

7. The population of nanoparticles of claim 6 wherein the amorphous drug core comprises an amorphous drug core that is at least about 99.9% w/w amorphous.

8. The population of nanoparticles of claim 1, wherein the period is of at least six months.

9. The population of nanoparticles of claim 8, wherein the period is of at least twelve months.

10. The population of nanoparticles of claim 9, wherein the period is of at least eighteen months.

11. The population of nanoparticles of claim 10, wherein the period is of at least twenty-four months.

12. The population of nanoparticles of claim 1, wherein the amorphous drug cores that are substantially free of dopant comprise amorphous drug cores containing less than about 15 weight percent dopant, wherein the weight percentage is based on the total weight of the amorphous drug core.

13. The population of nanoparticles of claim 12, wherein the amorphous drug cores that are substantially free of dopant comprise amorphous drug cores containing less than about 10 weight percent dopant, wherein the weight percentage is based on the total weight of the amorphous drug core.

14. The population of nanoparticles of claim 13, wherein the amorphous drug cores that are substantially free of dopant comprise amorphous drug cores containing less than about 5 weight percent dopant, wherein the weight percentage is based on the total weight of the amorphous drug core.

15. The population of nanoparticles of claim 14, wherein the amorphous drug cores that are substantially free of dopant comprise amorphous drug cores containing less than about 1 weight percent dopant; wherein the weight percentage is based on the total weight of the amorphous drug core.

16. The population of nanoparticles of claim 1, wherein the effective diameter of an amorphous drug core is less than or equal to about 1.5 micron.

17. The population of nanoparticles of claim 16, wherein the effective diameter of an amorphous drug core is less than or equal to about 1.0 micron.

18. The population of nanoparticles of claim 17, wherein the effective diameter of an amorphous drug core is less than or equal to about 0.75 micron.

19. The population of nanoparticles of claim 1, wherein the drug has a glass transition temperature greater than or equal to about 40 Deg. C.

20. The population of nanoparticles of claim 19, wherein the drug has a glass transition temperature greater than or equal to about 50 Deg. C.

21. The population of nanoparticles of claim 20, wherein the drug has a glass transition temperature greater than or equal to about 60 Deg. C.

22. The population of nanoparticles of claim 21, wherein the drug has a glass transition temperature greater than or equal to about 70 Deg. C.

23. The population of nanoparticles of claim 1, wherein the at least one stabilizer comprises co-stabilizers.

24. The population of nanoparticles of claim 1, wherein the at least one stabilizer is selected from polyvinylpyrrolidone; cellulosic polymers; copolymers of vinyl pyrrolidone and vinyl acetate; poloxamers; polyethylene glycols; polyvinyl alcohol; tyloxapol; polyoxyethylene castor oil derivatives; colloidal silicon dioxide; carbomers; CMC Na; Polysobates; benzalkonium chloride; charged phospholipids; sodium docusate; hydroxypropylmethyl cellulose; dioctyl sodium sulfosuccinate; gelatin; casein; lysozyme; albumin; cholesterol; stearic acid; calcium stearate; glycerol monostearate; sodium dodecylsulfate; methylcellulose; noncrystalline cellulose; magnesium aluminium silicate; alkyl aryl polyether sulfonates, and combinations thereof.

25. The population of nanoparticles of claim 1, wherein the water solubility of the drug is less than or equal to about 0.1 mg/ml at 25 Deg. C.

26. The population of nanoparticles of claim 25, wherein the water solubility of the drug is less than or equal to about 0.01 mg/ml at 25 Deg. C.

27. The population of nanoparticles of claim 26, wherein the water solubility of the drug is less than or equal to about 1 microgram/ml at 25 Deg. C.

28. A method of making a population of nanoparticles comprising:
forming amorphous drug cores with an effective diameter less than or equal to about 2.0 microns, wherein the amorphous drug cores are substantially free of dopant, and wherein the amorphous drug cores comprise a drug with properties that satisfy the following relationships:
a glass transition temperature greater than or equal to about 30 Deg. C.; and
water solubility at 25 Deg. C. less than or equal to about 1 mg/ml; and
adsorbing at least one stabilizer on a surface of the amorphous drug cores;
wherein the at least one stabilizer is present in an amount effective to provide an amorphous stability of the population of nanoparticles that is approximately equal to or greater than an amorphous stability of an amorphous bulk drug substance comprising the drug, as measured over a period of at least four months.

29. The method of claim 28, wherein forming amorphous drug cores comprises forming an amorphous bulk drug substance.

30. The method of claim 29, wherein forming an amorphous bulk drug substance comprises chemical synthesizing, melting/quenching the drug, solvent casting the drug, super critical fluid extraction, rapid precipitation by antisolvent addition, grinding/milling, freeze drying, spray freezing, solvent extraction, dehydration of hydrated compounds, freeze-drying, spray-drying, or combinations thereof.

31. The method of claim 29, wherein forming amorphous drug cores comprises nanosizing the amorphous bulk drug substance.

32. The method of claim 28, wherein nanosizing the amorphous bulk drug substance comprises milling, high speed homogenization, hydrodynamic cavitation, ultrasonication, or combinations thereof.

33. The method of claim 28, wherein the amorphous drug core comprises an amorphous drug core that is substantially amorphous.

34. The method of claim 33, wherein the amorphous drug core comprises an amorphous drug core that is at least about 95% w/w amorphous.

35. The method of claim 34, wherein the amorphous drug core comprises an amorphous drug core that is at least about 98% w/w amorphous.

36. The method of claim 35, wherein the amorphous drug core comprises an amorphous drug core that is at least about 99% w/w amorphous.

37. The method of claim 36, wherein the amorphous drug core comprises an amorphous drug core that is at least about 99.5% w/w amorphous.

38. The method of claim 37, wherein the amorphous drug core comprises an amorphous drug core that is at least about 99.9% w/w amorphous.

39. The method of claim 28, wherein the period is of at least six months.

40. The method of claim 39, wherein the period is of at least twelve months.

41. The method of claim 40, wherein the period is of at least eighteen months.

42. The method of claim 42, wherein the period is of at least twenty-four months.

43. The method of claim 28, wherein the amorphous drug cores that are substantially free of dopant comprise amorphous drug cores containing less than about 15 weight percent dopant, wherein the weight percentage is based on the total weight of the amorphous drug core.

44. The method of claim 43, wherein the amorphous drug cores that are substantially free of dopant comprise amorphous drug cores containing less than about 10 weight percent dopant, wherein the weight percentage is based on the total weight of the amorphous drug core.

45. The method of claim 44, wherein the amorphous drug cores that are substantially free of dopant comprise amorphous drug cores containing less than about 5 weight percent dopant, wherein the weight percentage is based on the total weight of the amorphous drug core.

46. The method of claim 45, wherein the amorphous drug cores that are substantially free of dopant comprise amorphous drug cores containing less than about 1 weight percent dopant; wherein the weight percentage is based on the total weight of the amorphous drug core.

47. The method of claim 28, wherein the effective diameter of an amorphous drug core is less than or equal to about 1.5 micron.

48. The method of claim 47, wherein the effective diameter of an amorphous drug core is less than or equal to about 1.0 micron.

49. The method of claim 48, wherein the effective diameter of an amorphous drug core is less than or equal to about 0.75 micron.

50. The method of claim 28, wherein the drug has a glass transition temperature greater than or equal to about 40 Deg. C.

51. The method of claim 50, wherein the drug has a glass transition temperature greater than or equal to about 50 Deg. C.

52. The method of claim 51, wherein the drug has a glass transition temperature greater than or equal to about 60 Deg. C.

53. The method of claim 52, wherein the drug has a glass transition temperature greater than or equal to about 70 Deg. C.

54. The method of claim 28, wherein the at least one stabilizer comprises co-stabilizers.

55. The method of claim 28, wherein the at least one stabilizer is selected from polyvinylpyrrolidone; cellulosic polymers; copolymers of vinyl pyrrolidone and vinyl acetate; poloxamers; polyethylene glycols; polyvinyl alcohol; tyloxapol; polyoxyethylene castor oil derivatives; colloidal silicon dioxide; carbomers; CMC Na; Polysobates; benzalkonium chloride; charged phospholipids; sodium docusate; hydroxypropylmethyl cellulose; dioctyl sodium sulfosuccinate; gelatin; casein; lysozyme; albumin; cholesterol; stearic acid; calcium stearate; glycerol monostearate; sodium dodecylsulfate; methylcellulose; noncrystalline cellulose; magnesium aluminium silicate; alkyl aryl polyether sulfonates, and combinations thereof.

56. The method of claim 28, wherein the water solubility of the drug is less than or equal to about 0.1 mg/ml at 25 Deg. C.

57. The method of claim 56, wherein the water solubility of the drug is less than or equal to about 0.01 mg/ml at 25 Deg. C.

58. The method of claim 57, wherein the water solubility of the drug is less than or equal to about 1 microgram/ml at 25 Deg. C.

59. The method of claim 29, wherein the amorphous bulk drug substance is at least about 80% w/w amorphous.

60. The method of claim 59, wherein the amorphous bulk drug substance is at least about 85% w/w amorphous.

61. The method of claim 60, wherein the amorphous bulk drug substance is at least about 90% w/w amorphous.

62. The method of claim 61, wherein the amorphous bulk drug substance is at least about 95% w/w amorphous.

63. The method of claim 62, wherein the amorphous bulk drug substance is at least about 99% w/w amorphous.

64. The method of claim 63, wherein the amorphous bulk drug substance is at least about 99.5% w/w amorphous.
